(19) United States
(12) Patent Application Publication
(10) Pub. No.: US 2012/0208899 A1
(43) Pub. Date: Aug. 16, 2012

(54) Tool for Assessment of Symptoms of Irritable Bowel Syndrome

(75) Inventors: Leticia Delgado-Herrera, Lake Forest, IL (US); Abhijit Barve, Bangalore (IN); Kathryn Lasch, West Newton, MA (US); Allam Fakhoury, Algonquin, IL (US); Smita Kothari, Jersey City, NJ (US); Akito Nishida, Saitama City (JP); Patrick Marquis, Newton, MA (US); Glen Spears, Third Lake, IL (US); Bernhardt G. Zeiher, Saline, MI (US); Kathleen Rees Rosa, Wilmington, NC (US); Laura Tesler Waldman, Norfolk, MA (US)

(73) Assignee: Astellas US LLC, Deerfield, IL (US)

(21) Appl. No.: 13/274,040

(22) Filed: Oct. 14, 2011

Related U.S. Application Data
(63) Continuation-in-part of application No. PCT/US10/31502, filed on Apr. 16, 2010.
(60) Provisional application No. 61/498,377, filed on Jun. 17, 2011.
(60) Provisional application No. 61/319,656, filed on Mar. 31, 2010.
(60) Provisional application No. 61/170,059, filed on Apr. 16, 2009.

(51) Int. Cl. A61K 35/00; A61P 29/00; A61B 5/00; A61P 1/00

(52) U.S. Cl. 514/789; 600/300

(57) ABSTRACT

Methods of treating and/or diagnosing irritable bowel syndrome (IBS) are provided, wherein treatment or diagnosis is undertaken on the basis of the patient's recordation of events associated with IBS in an event log and/or symptom diary.

### INCORPORATION BY REFERENCE TO OTHER PATENT APPLICATIONS

This patent application is a continuation-in-part of International Patent Application No. PCT/US2010/031502, filed Apr. 16, 2010, which claims the benefit of U.S. Provisional Patent Application No. 61/170,059, filed Apr. 16, 2009, and U.S. Provisional Patent Application No. 61/319,656, filed Mar. 31, 2010; this patent application also claims the benefit of U.S. Provisional Patent Application No. 61/498,377, filed Jun. 17, 2011, each of which is incorporated herein by reference.

### BACKGROUND OF THE INVENTION

About 10% to about 20% of adults and adolescents worldwide suffer from irritable bowel syndrome (IBS). IBS exacts a disproportionate share of the US health care dollar. In US managed-care populations, for example, total direct costs incurred by subjects suffering from IBS have been measured to amount to about 50% higher than those not suffering from IBS. The negative impact of IBS on patients, their families, and their friends can be great, especially in moderate to severe IBS-D, which accounts for extremely high utilization of health care resources and impairment of quality-of-life.

According to the Rome III criteria, IBS is diagnosed with recurrent abdominal pain or discomfort at least three days per month in the last three months associated with two or more of the following: (1) improvement with defecation, (2) onset associated with a change in frequency of stool, and/or (3) onset associated with a change in form (appearance) of stool. It is also noted that these symptoms typically have had symptom onset at least six months prior to diagnosis. In addition, according to the Rome III criteria, irritable bowel syndrome with diarrhea or irritable bowel syndrome, diarrhea predominant (both abbreviated “IBS-D”) is defined as loose mushy stool at least 25% of bowel movements and hard or lumpy stool in less than 25% of bowel movements. However, there currently is no well-validated symptom severity scales for IBS-D.

Multidisciplinary approaches (including diet changes, over-the-counter (OTC) products, and prescription medications) for treating IBS patients are frequent, and such drugs and remedies are often prescribed by family practitioners. Current treatment is mostly based on pharmacological therapy and is palliative in nature. Only one prescription medication—Alosetron, a selective 5-HT3 antagonist—has been approved by the US Food and Drug Administration (FDA) for IBS-D, and it is only available under a restricted access program due to risks of severe side effects if taken by patients who suffer from any other form of IBS (e.g., constipation-predominant irritable bowel syndrome (IBS-C) and mixed IBS).

An instrument (tool) developed and validated for the IBS patient population, ideally to assess symptom severity, would represent an advancement in the treatment of IBS.

### BRIEF SUMMARY OF THE INVENTION

The invention provides a tool for assessment of symptoms of irritable bowel syndrome in a human patient. The tool comprises a diary and/or event log, suitable for psychometric testing of symptoms of IBS. The symptom diary and event log can be used singly or in combination. The symptom diary and/or event log can be used to assess the progress of treatment of IBS in a patient.

In one aspect, the invention provides a method for diagnosing IBS comprising (a) providing a patient with an event log, wherein the patient is instructed to record in the event log the date and time of each bowel movement together with a description thereof; and/or (b) providing the patient with a symptom diary, wherein the patient is instructed to complete the symptom diary by selecting a characterization from a series of pre-determined characterizations regarding the patient's subjective experience of events associated with IBS; (c) reviewing the event log and/or symptom diary completed by the patient; and (d) determining whether a diagnosis of irritable bowel syndrome is appropriate.

In another aspect, the invention provides a method for treating IBS comprising (a) administering to a patient having IBS an IBS treatment at a dosage and frequency; (b) providing the patient with an event log, wherein the patient is instructed to record in the event log the date and time of each bowel movement together with a description thereof; and/or (c) providing the patient with a symptom diary, wherein the patient is instructed to complete the symptom diary by selecting a characterization from a series of pre-determined characterizations regarding the patient's subjective experience of events associated with IBS; (d) reviewing the event log and/or symptom diary completed by the patient to assess changes in symptoms; (e) revising the dosage and frequency of the IBS treatment, or, optionally, administering a different IBS treatment; and (f) repeating steps (a)-(e) until the patient achieves a clinically satisfactory improvement in IBS symptoms.

In another aspect, the invention provides calculating an index value based on the patient's score in each category of the symptom diary and/or event log, wherein the index value indicates the relative severity of a patient's IBS symptoms as compared to a healthy individual.

The IBS can be any type of IBS, such as IBS-D. Also, the tool can be used in conjunction with therapy for IBS (e.g., dietary changes, medication, or both), i.e., in which the patent is undergoing treatment for IBS. In this sense, the event log, symptom diary, or both, can provide meaningful cognitive feedback concerning the severity of the IBS symptoms in response to therapy. In a preferred embodiment, the patient is undergoing treatment with a prescription medication approved for treating IBS or under investigation for such indication.

### BRIEF DESCRIPTION OF THE SEVERAL VIEWS OF THE DRAWING(S)

FIG. 1A depicts page 1 of an exemplary 10-question symptom diary to be provided to a patient.

FIG. 1B depicts page 2 of an exemplary 10-question symptom diary to be provided to a patient.

FIG. 2 depicts an exemplary event log to be provided to a patient.

FIG. 3A depicts page 1 of an exemplary 15-question symptom diary to be provided to a patient.

FIG. 3B depicts page 2 of an exemplary 15-question symptom diary to be provided to a patient.

FIG. 3C depicts page 3 of an exemplary 15-question symptom diary to be provided to a patient.

FIG. 4 depicts an exemplary event log to be provided to a patient.

FIG. 5A depicts page 1 of an exemplary 17-question symptom diary to be provided to a patient.

FIG. 5B depicts page 2 of an exemplary 17-question symptom diary to be provided to a patient.

FIG. 5C depicts page 3 of an exemplary 17-question symptom diary to be provided to a patient.

FIG. 6 depicts an exemplary event log to be provided to a patient.

FIG. 7 depicts an exemplary event log to be provided to a patient.

FIG. 8 (8A and 8B) depict an exemplary 6-question symptom diary to be provided to a patient.

FIG. 9 depicts an exemplary event log to be provided to a patient.

FIG. 10 depicts an exemplary event log to be provided to a patient.

FIG. 11 (11A and 11B) depict an exemplary 7-question symptom diary to be provided to a patient.

FIG. 12 (12A and 12B) depict an exemplary 7-question symptom diary to be provided to a patient.

### DETAILED DESCRIPTION OF THE INVENTION

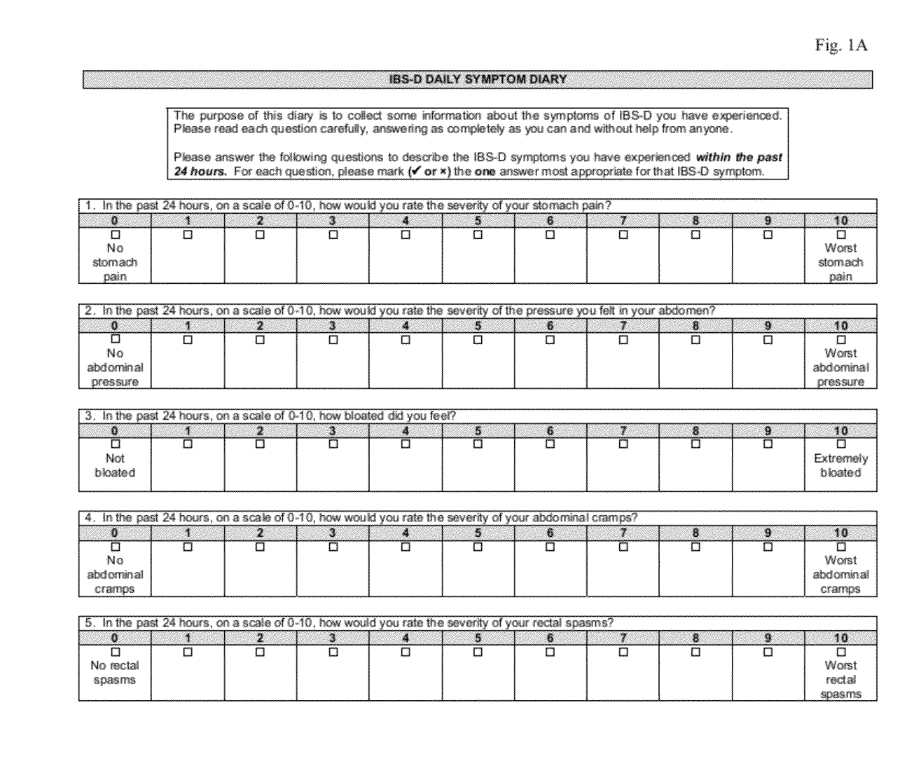

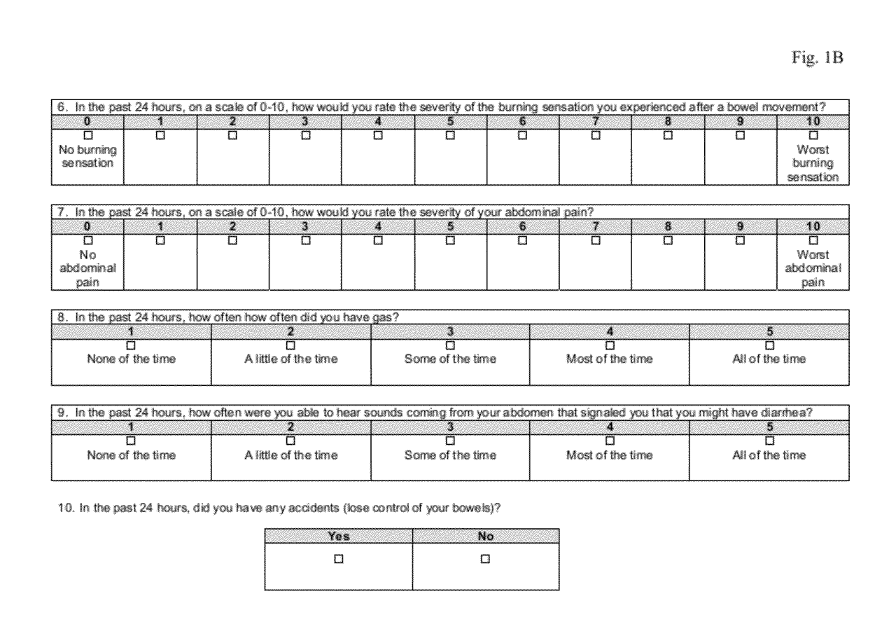

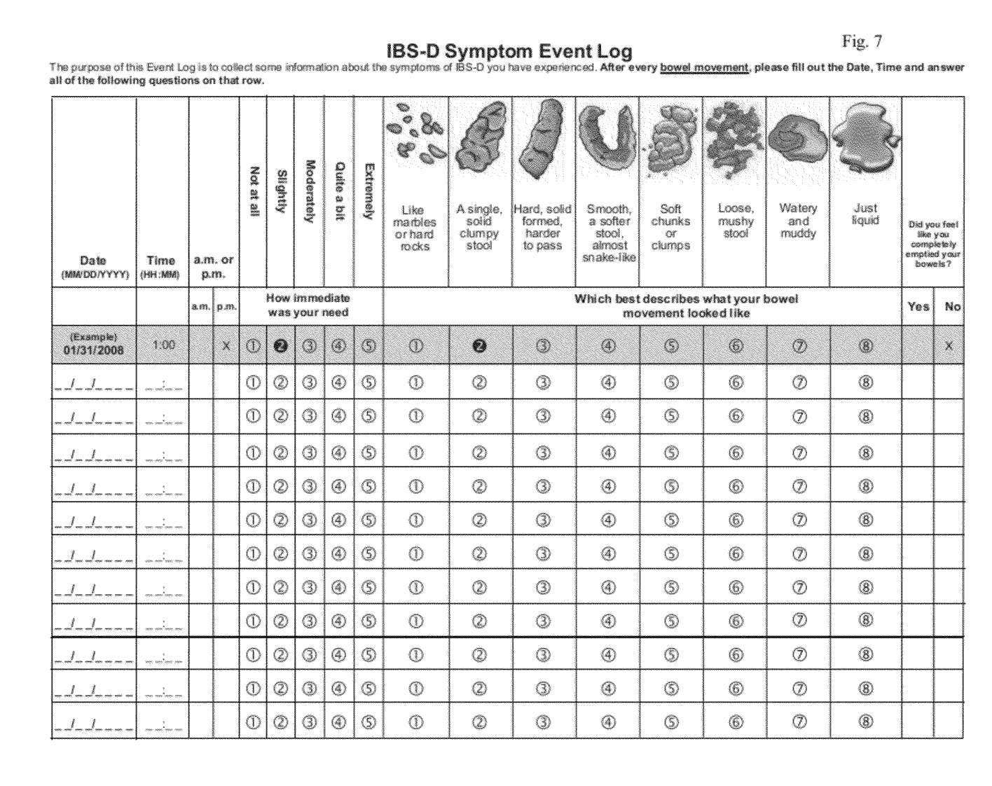

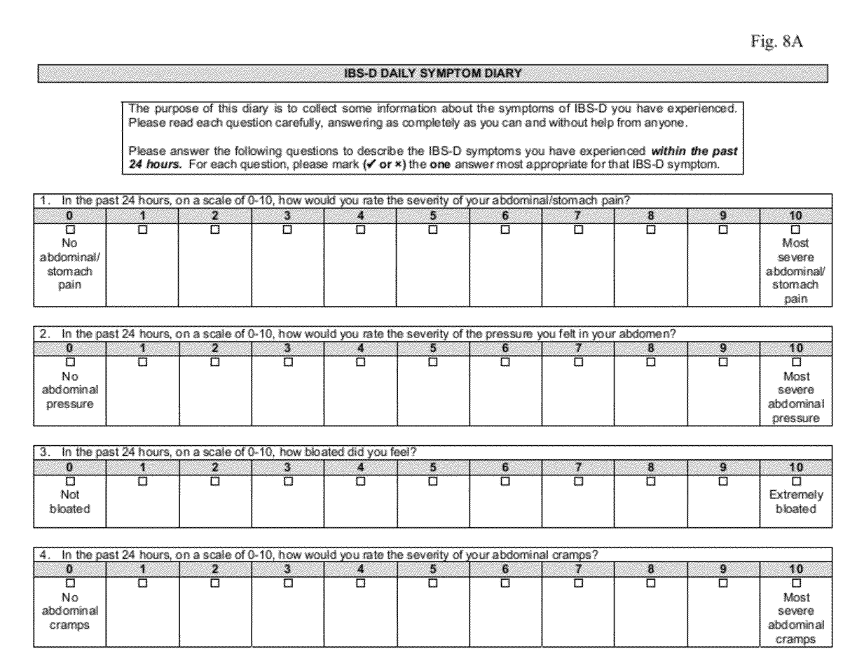

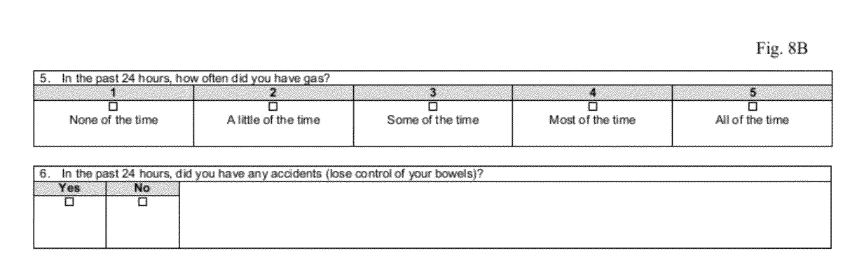

The invention provides methods of using the symptom diary and/or event log, such as provided in FIGS. 1-8, in treating or diagnosing IBS, or in assessing a patient's symptoms or response to IBS treatment. A preferred symptom diary is set forth at FIGS. 8A and 8B, and a preferred event log is set forth at FIG. 7.

In one aspect, the invention provides a method for diagnosing IBS comprising (a) providing a patient with an event log, wherein the patient is instructed to record in the event log the date and time of each bowel movement together with a description thereof; and/or (b) providing the patient with a symptom diary, wherein the patient is instructed to complete the symptom diary by selecting a characterization from a series of pre-determined characterizations regarding the patient's subjective experience of events associated with IBS; (c) reviewing the event log and/or symptom diary completed by the patient; and (d) determining whether a diagnosis of irritable bowel syndrome is appropriate. In preferred embodiments, the patient is provided with both an event log and a symptom diary. However, in some embodiments, the patient can be provided with an event log and not a symptom diary, or a symptom diary and not an event log.

In another aspect, the invention provides a method for treating IBS comprising (a) administering to a patient having IBS an IBS treatment at a dosage and frequency; (b) providing the patient with an event log, wherein the patient is instructed to record in the event log the date and time of each bowel movement together with a description thereof; and/or (c) providing the patient with a symptom diary, wherein the patient is instructed to complete the symptom diary by selecting a characterization from a series of pre-determined characterizations regarding the patient's subjective experience of events associated with IBS; (d) reviewing the event log and/or symptom diary completed by the patient; (e) revising the dosage and frequency of the IBS treatment, or, optionally, administering a different IBS treatment; and (f) repeating steps (a)-(e) until the patient achieves a clinically satisfactory improvement in IBS symptoms. In preferred embodiments, the patient is provided with both an event log and a symptom diary. However, in some embodiments, the patient can be provided with an event log and not a symptom diary, or a symptom diary and not an event log.

In embodiments in which the patient is provided with an event log, the patient can be instructed to record bowel movements over a specified time period. For example, the patient can be instructed to record bowel movements over one, two, three, or four weeks. Alternatively, the patient can be instructed to record bowel movements over one or multiple months. In some embodiments, such as when the patient is undergoing investigational drug therapy, it may be preferred that the patient records bowel movements for the full duration of the investigational drug therapy.

The event log can have any form useful to the clinician. However, in preferred embodiments, the event log includes one or more of the questions provided in Table 4 or Table 5 of Example 1 or similar questions, along with pre-determined characterizations from which the patient can select. In a more preferred embodiment, the event log includes each question provided in Table 4 or Table 5. In a most preferred embodiment, the event log comprises the form of FIG. 7.

In embodiments in which the patient is provided with a symptom diary, the patient can be instructed to select a characterization from a series of pre-determined characterizations regarding the patient's subjective experience of events associated with IBS. The patient can be instructed to complete the symptom diary using characterizations of the patient's experience of events associated with IBS on a single day. Alternatively, the patient can be instructed to complete multiple symptom diaries over a specified time period, wherein each symptom diary is completed using characterizations of the patient's experience of events associated with IBS on a different day. For example, the patient can be instructed to complete symptom diaries each day for one, two, three, or four weeks. Alternatively, the patient can be instructed to complete daily symptom diaries over one or multiple months. In some embodiments, such as when the patient is undergoing investigational drug therapy, it may be preferred that the patient completes daily symptom diaries for the full duration of the investigational drug therapy.

The symptom diary can have any form useful to the clinician. However, in preferred embodiments, the symptom diary includes one or more of the questions provided in Table 3 of Example 1 or FIG. 8, along with pre-determined characterizations from which the patient can select. In preferred embodiments, the symptom diary comprises each question provided in Table 3 or FIG. 8. In a most preferred embodiment, the symptom diary comprises the form provided in FIG. 1 (1A and 1B) or FIG. 8 (8A and 8B).

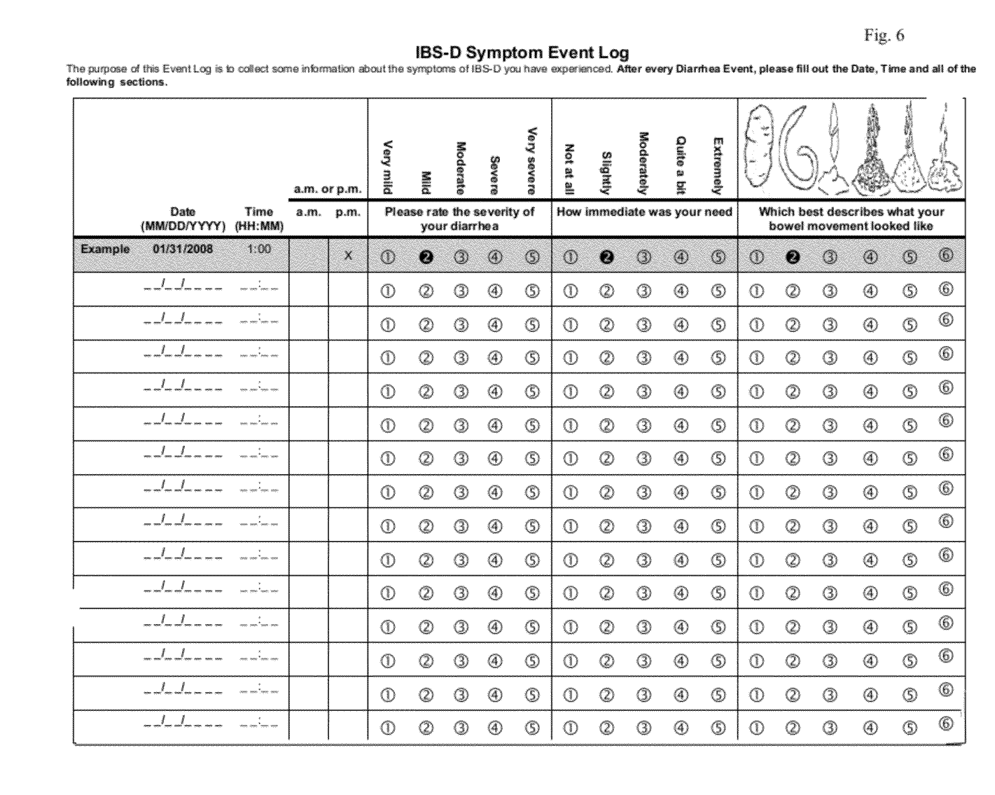

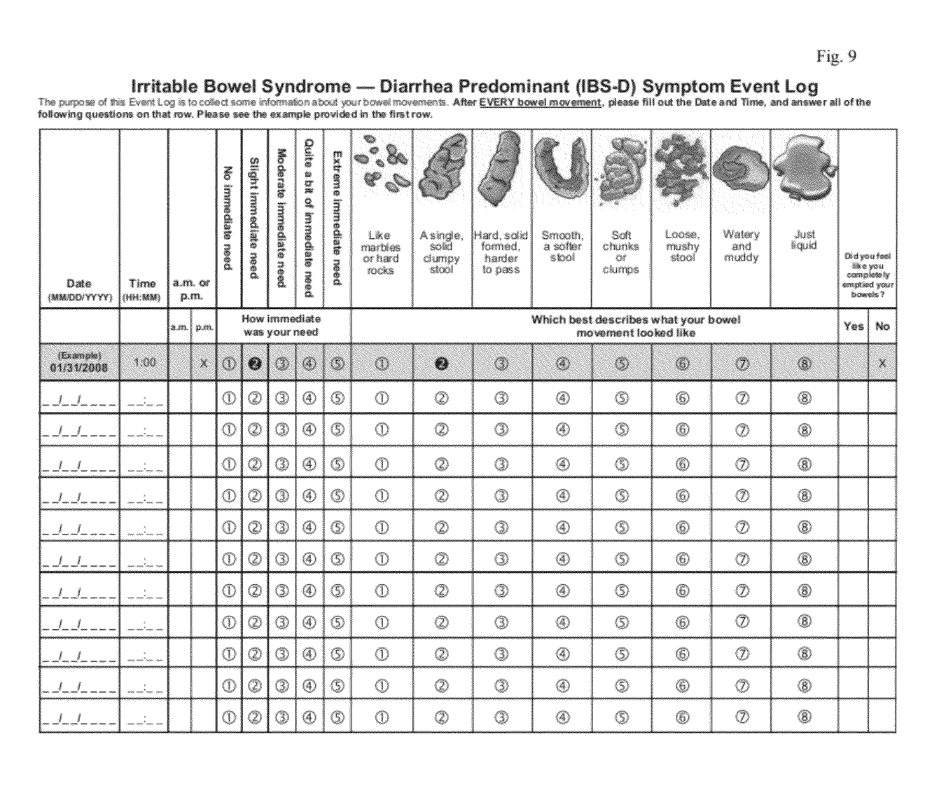

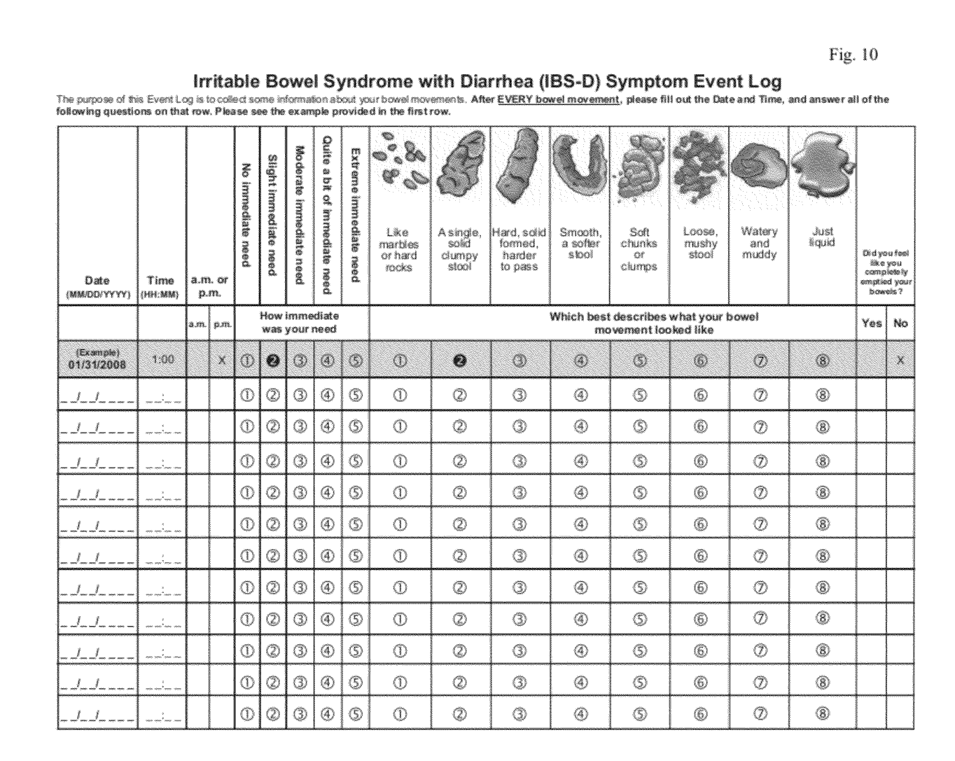

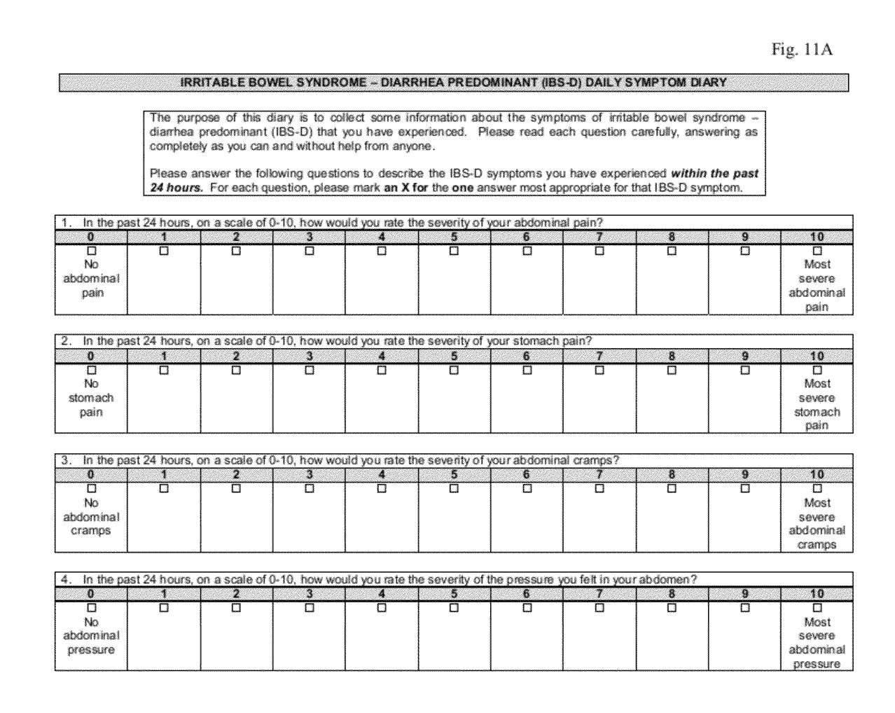

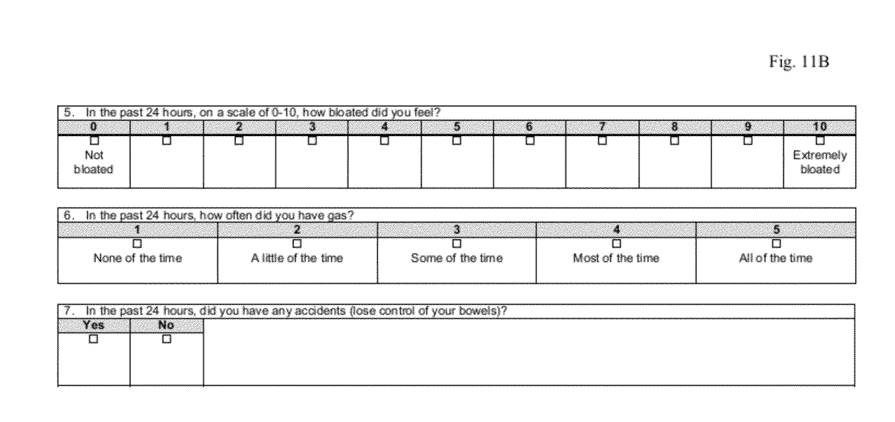

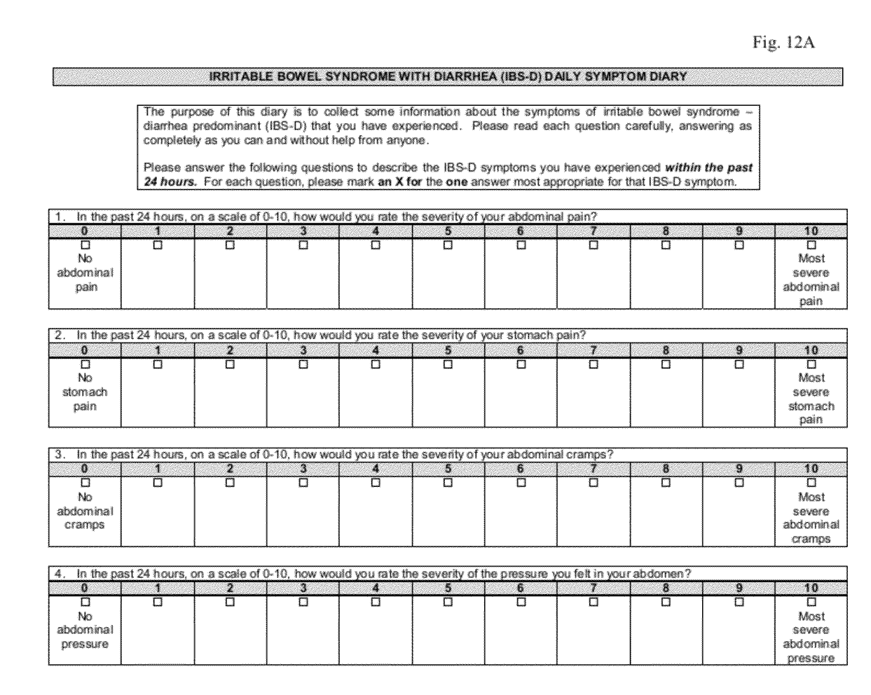

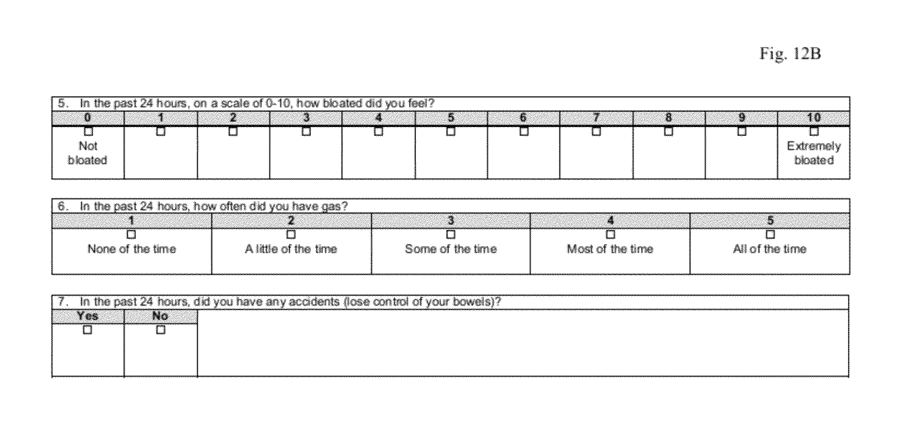

It will be understood that the event log and symptom diary are intended to record a patient's experience. Therefore, any suitable format providing any number of pre-determined characterizations can be provided for the patient's answers. In some embodiments, it will be useful to provide pre-determined characterizations for the symptom diary and event log, for example as shown in Tables 3 or FIG. 8 and Tables 4 or 5, respectively. However, as examples, the pre-determined characterizations can be based on a 5-point scale (Likert scale), an 11-point scale (0-10), or a positive/negative scale (yes/no). In responding to questions regarding the appearance of the bowel movement, the event log desirably provides visual and/or text characterizations. One such scale can be based on the Adapted Bristol Stool Form Scale (shown, for example, in FIG. 6), which, for IBS-D, can be modified to include forms relevant to IBS-D (e.g., excluding very hard stool forms). Other such scales are as depicted in FIGS. 7, 9, and 10.

In some embodiments, the patient is undergoing a treatment for IBS. The treatment can be any IBS treatment, such as a pharmaceutical or biologic treatment, or a dietary therapy, or other therapy. In some embodiments, the therapy is a prescription medication approved or under investigational use for treatment of IBS.

In another aspect, the invention provides calculating an index value based on the patient's score in each category of the symptom diary and event log, wherein the index value indicates the relative severity of a patient's IBS symptoms as compared to a healthy individual or to a previous baseline. The index value can also be used to determine whether a patient has achieved clinically satisfactory improvement in IBS symptoms. The index value can be calculated by any method known to one of skill in the art and/or deemed to be useful in applying the methods of the present invention. However, it is not necessary to calculate a single index value in order to determine whether a patient has achieved improvement in IBS symptoms. If the patient shows improvement based on the total score of six or more items of the symptom diary, in general, such improvement will be considered clinically satisfactory improvement in IBS symptoms.

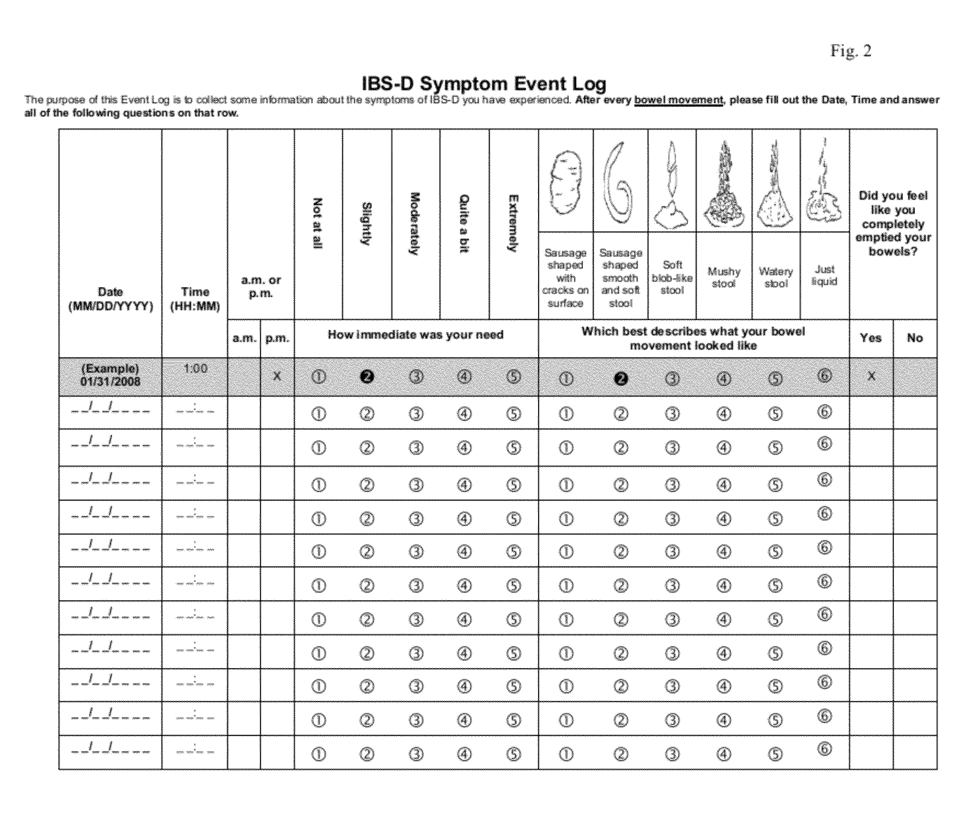

Additionally, the symptom diary and event log can be used to assess primary and secondary endpoints for diagnosing patients as having IBS or for evaluating the progress of treatment of IBS patients. For IBS-D, diarrhea (stool frequency and stool consistency) and pain are the most frequent and most bothersome symptoms of IBS-D, and these can serve as co-primary endpoints. For example, a patient reporting a weekly average abdominal pain severity over a 24 hour period of a threshold value (e.g., 3 or higher, 4 or higher, 5 or higher, etc. on a 0-10 point scale) and a weekly average stool consistency of a threshold value (for example, 4 or above according to the scale presented in FIG. 2, or 5 or 6 and above according to the scale presented in FIGS. 7, 9 and 10) could on that basis be diagnosed as having IBS-D. To assess a patient's response to therapy for IBS-D, a patient can be considered responding if, for example, the patient experiences a threshold decrease (such as, for example, 25% or greater, 30% or greater, 35% or greater) in the weekly average of abdominal pain severity over a 24 hour period. Successful response to IBS-D treatment also can be indicated by a patient who experiences a improved weekly average stool consistency relative to a baseline measurement, or a weekly average stool consistency below a threshold value (for example, 3 or below according to the scale presented in FIG. 2, or 4 or 5 and below according to the scale presented in FIGS. 7, 9, and 10). Preferably, improvement in both abdominal pain severity and stool consistency are considered. Further, for IBS-D, immediate need to have a bowel movement, bloating, pressure, gas, incomplete evacuation, and rectal symptoms can serve as secondary endpoints, and improvement in these categories can also be employed to assess an IBS-D patient's response to treatment.

The following examples further illustrate the invention but, of course, should not be construed as in any way limiting its scope.

### Example 1

This example demonstrates a method for developing a tool for assessment of symptoms of Irritable Bowel Disease.

Two sets of concept elicitation focus groups were conducted, with patients in each set meeting the ROME III inclusion and exclusion criteria for IBS-D. In the first (Set 1), 34 patients were interviewed in 8 concept elicitation focus groups having 2-9 subjects per group. In the second (Set 2), 32 patients under 70 years of age were interviewed in four gender-specific concept elicitation focus groups (n=3 female, n=1 male). Demographic and health information for each patient was recorded and is presented in Table 1.

TABLE 1
Summary of Socio-Demographic and Health Information

| Criteria | Set 1 | | Set 2 | | Overall | |
|---|---|---|---|---|---|---|
| Number | 34 | | 32 | | 64 | |
| Female (%) | 22 | (64.71) | 24 | (75.00) | 46 | (71.88) |
| Age Mean ± SD | 44.65 ± 15.5 | | 45 ± 10.84 | | 45.02 ± 13.35 | |
| (Range) | (23, 69) | | (21, 68) | | (21, 69) | |
| Ethnicity (%) | | | | | | |
| Asian | 3 | (8.82%) | N/A | | 3 | (4.69%) |
| Black/African | 4 | (11.76%) | 4 | (12.50%) | 8 | (12.50%) |
| American | | | | | | |
| Hispanic/Latino | 1 | (2.94%) | 1 | (3.13%) | 2 | (3.13%) |
| (of any race) | | | | | | |
| White/Caucasian | 26 | (76.47%) | 26 | (81.25%) | 52 | (81.25%) |
| Multiracial | N/A | | 1 | (3.13%) | 1 | (1.56%) |
| Level of education (%) | | | | | | |
| High school diploma | 5 | (14.71%) | 6 | (18.75%) | 11 | (17.19%) |
| or GED | | | | | | |
| Some college | 6 | (17.65%) | 13 | (40.63%) | 19 | (29.69%) |
| Vocational school or | 2 | (5.88%) | 5 | (15.63%) | 7 | (10.94%) |
| certificate program | | | | | | |
| College or university | 16 | (47.06%) | 6 | (18.75%) | 22 | (34.38%) |
| degree (2- or 4- year) | | | | | | |
| Graduate degree | 5 | (14.71%) | 2 | (6.25%) | 7 | (10.94%) |
| Work status (%) | | | | | | |
| Working full-time | 15 | (44.12) | NE | | NE | |
| Working part-time | 7 | (20.59) | NE | | NE | |
| Homemaker | 3 | (8.82) | NE | | NE | |
| Student | 2 | (5.88) | NE | | NE | |
| Retired | 8 | (23.53) | NE | | NE | |
| Unemployed | 1 | (2.94) | NE | | NE | |
| Health in general/ | | | | | | |
| health status (%) | | | | | | |
| Excellent | 4 | (11.76%) | N/A | | 4 | (6.25%) |
| Very good | 9 | (26.47%) | 6 | (18.75%) | 15 | (23.44%) |
| Good | 14 | (41.18%) | 20 | (62.50%) | 34 | (53.13%) |
| Fair | 5 | (14.71%) | 6 | (18.75%) | 11 | (17.19%) |
| Poor | 2 | (5.88%) | N/A | | 2 | (3.13%) |
| Severity of IBS-D (%) | | | | | | |
| Very mild | 4 | (11.76%) | 1 | (3.13%) | 5 | (7.81%) |
| Mild | 8 | (23.53%) | 4 | (12.50%) | 12 | (18.75%) |
| Moderate | 19 | (55.88%) | 18 | (56.25%) | 37 | (57.81%) |
| Severe | 3 | (8.82%) | 8 | (25.00%) | 11 | (17.19%) |

| | | | | | |
|---|---|---|---|---|---|
| Very severe (%) | 0 | 1 | (3.13%) | 1 | (1.56%) |

GED: General Educational Development;
IBS-D: diarrhea-predominant irritable bowel syndrome ;
N/A: not applicable;
NE: not evaluated

Grounded theory data collection and analysis methods were used, including constant comparison of quotations by participants, to determine whether concepts were simple or complex; to determine the grouping of sub-concepts; to determine concepts and domains; to determine the recall period; to determine the response options (e.g., a severity or frequency item); to determine whether the items emphasizing patients' exact words were appropriate; and to develop the conceptual framework.

In Set 1, thirty-six different concepts were elicited, with 29 concepts saturated. Responses from Set 2 were compared with those of Set 1, and determined to be similar. Ethnicity and other demographic data did not impact the reporting of the symptoms by the patients. In particular, educational level did not impact how the symptoms were reported by patients. For example, all patients tend to describe the occurrence of diarrhea (in relation to eating), and description of diarrhea (“the runs”) similarly, as illustrated in Table 2.

TABLE 2

| Level of Education | Sample Quotes |
|---|---|
| High school diploma | . . . overeating. Sometimes like a holiday - |
| or GED | Thanksgiving . . . I'll get the diarrhea, got |
| | to empty my system. |
| | . . . I got the runs . . . |
| Vocational school or | . . . maybe the food that I was eating . . . the |
| certificate program | runs. |
| | . . . get the runs . . . |
| At least some college, | . . . as soon as you eat something . . . immedi- |
| college degree or higher | ately have to go to the restroom . . . like every |
| | time I eat something. |
| | Just got the runs . . . had the runs when I drank |
| | coffee. |

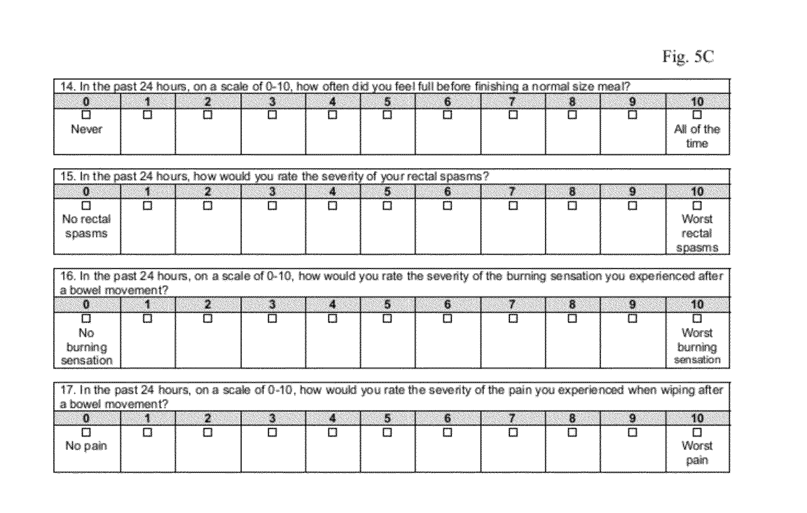

Concepts elicited that were mentioned spontaneously and that were saturated were included in a draft symptom diary (FIG. 5) and a draft event log (FIG. 6). However, some concepts that met these criteria were excluded due to reasons such as a lack of clinical relevance or specificity to IBS-D, such as vomiting, nausea, heartburn, and sweating. More specifically, concepts included in the draft symptom diary and draft event log were limited to lower GI symptoms in accordance with the ROME III Criteria and clinical diagnosis of IBS-D.

The concepts that were retained for use in the draft symptom diary and event log were: Diarrhea, immediate need (urgency), bloating/pressure, frequency of bowel movement, cramps, abdominal/stomach pain, gas, complete emptying of bowels/incomplete evacuation, accident, bubbling in intestines/bowel sounds, rectal burning, stool consistency, rectal spasm, and pain while wiping.

The draft symptom diary was developed to capture IBS-D symptoms over a given day while the draft event log was developed to report information regarding individual bowel movements.

Eleven cognitive interviews were conducted to determine the comprehensiveness, understanding, appropriateness, and readability of the draft symptom diary and draft event log. Patients were asked to complete the 17-item draft symptom diary (FIG. 5) and draft event log (FIG. 6), and to provide their feedback using the “think aloud” method. Patients were asked to identify words, terms, or concepts that they did not understand or interpreted differently than had been intended. They were also asked if revisions should be made to the 17-item draft symptom diary or event log to make them more appropriate, comprehensive, or interpretable. After the cognitive interviews, two questions based on concepts listed above were deleted from the 17-item draft symptom diary: “In the past 24 hours, how would you rate your immediate need to use the bathroom to have a bowel movement?” and “In the past 24 hours, how often did you feel full before finishing a normal size meal?”

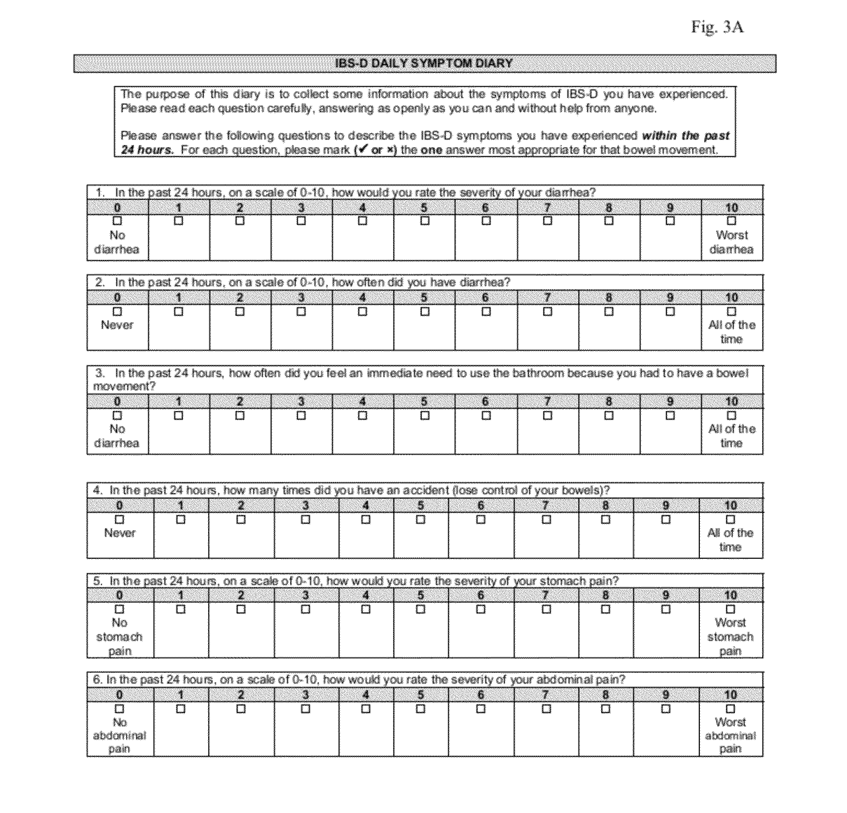

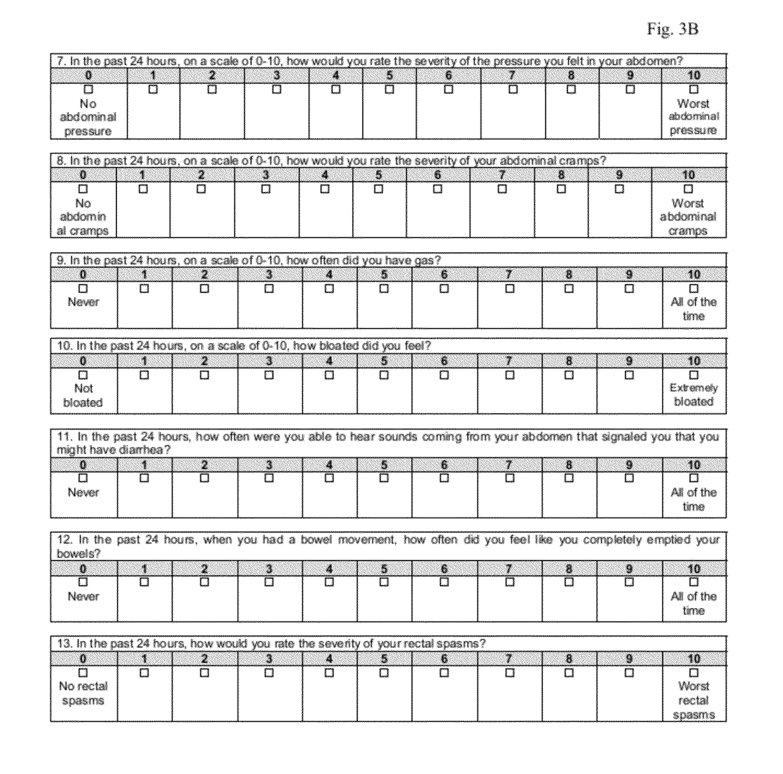

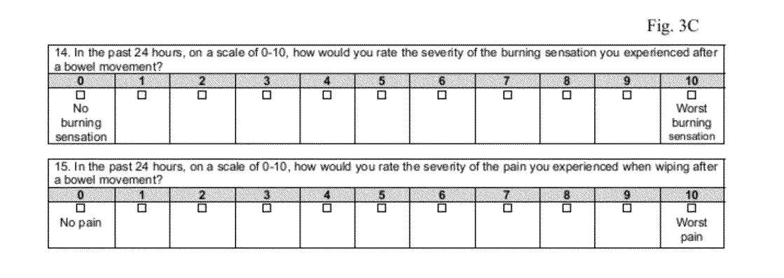

Five additional questions were deleted from the resulting 15-item symptom diary (FIG. 3) in order to avoid redundancy and optimize clarity. The remaining 10 questions of the symptom diary are presented in Table 3. A complete version of an exemplary symptom diary is presented in FIG. 1 (1A and 1B).

Another exemplary symptom diary is presented in FIG. 8 (8A and 8B). The version presented in FIG. 8 differs from that presented in FIG. 1 in that questions relating to impact symptoms (specifically those relating to rectal spasms, burning sensation, and abdominal sounds) have been removed, whereas the six questions relating to disease symptoms of IBS-D have been retained. The wording of certain questions and answers also varies slightly between the diary presented in FIG. 8 and FIG. 1.

TABLE 3
Symptom Diary

| Question | Provided responses |
|---|---|
| 1. In the past 24 hours, on a scale of 0 to 10, | A range of 0-10, with 0 characterized as “No |
| how would you rate the severity of your | stomach pain” and 10 characterized as |
| stomach pain? | “Worst stomach pain.” |
| 2. In the past 24 hours, on a scale of 0 to 10, | A range of 0-10, with 0 characterized as “No |
| how would you rate the severity of the | abdominal pressure” and 10 characterized as |
| pressure you felt in your abdomen? | “Worst abdominal pressure.” |
| 3. In the past 24 hours, on a scale of 0 to 10, | A range of 0-10, with 0 characterized as “Not |
| how bloated did you feel? | bloated” and 10 characterized as “Extremely |
| | bloated.” |
| 4. In the past 24 hours, on a scale of 0 to 10, | A range of 0-10, with 0 characterized as “No |
| how would you rate the severity of your | abdominal cramps” and 10 characterized as |
| abdominal cramps? | “Worst abdominal cramps.” |
| 5. In the past 24 hours, on a scale of 0 to 10, | A range of 0-10, with 0 characterized as “No |
| how would you rate the severity of your | rectal spasms” and 10 characterized as |
| rectal spasms? | “Worst rectal spasms.” |
| 6. In the past 24 hours, on a scale of 0 to 10, | A range of 0-10, with 0 characterized as “No |
| how would you rate the severity of the | burning sensation” and 10 characterized as |
| burning sensation you experienced after a | “Worst burning sensation.” |
| bowel movement? | |
| 7. In the past 24 hours, on a scale of 0 to 10, | A range of 0-10, with 0 characterized as “No |
| how would you rate the severity of your | abdominal pain” and 10 characterized as |
| abdominal pain? | “Worst abdominal pain.” |
| 8. In the past 24 hours, how often did you | A 5-point Likert scale: (1) None of the time; |
| have gas? | (2) A little of the time; (3) Some of the time; |
| | (4) Most of the time; (5) All of the time. |
| 9. In the past 24 hours, how often were you | A 5-point Likert scale: (1) None of the time; |
| able to hear sounds coming from your | (2) A little of the time; (3) Some of the time; |
| abdomen that signaled that you might have | (4) Most of the time; (5) All of the time. |
| diarrhea? | |
| 10. In the past 24 hours did you have any | Yes or No. |
| accidents (lose control of your bowels)? | |

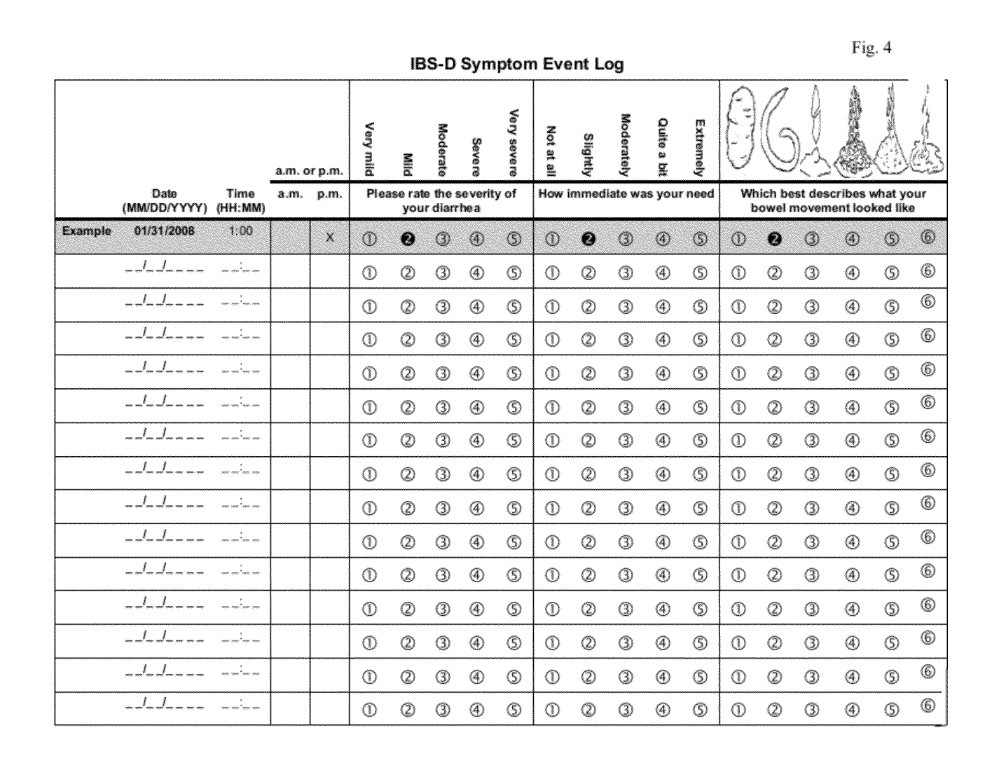

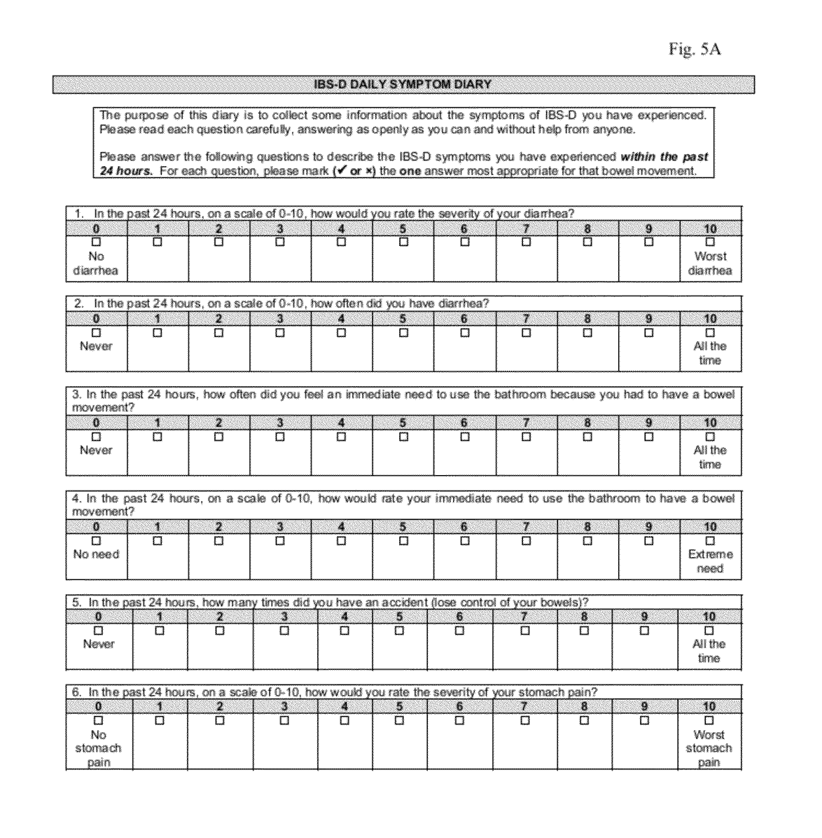

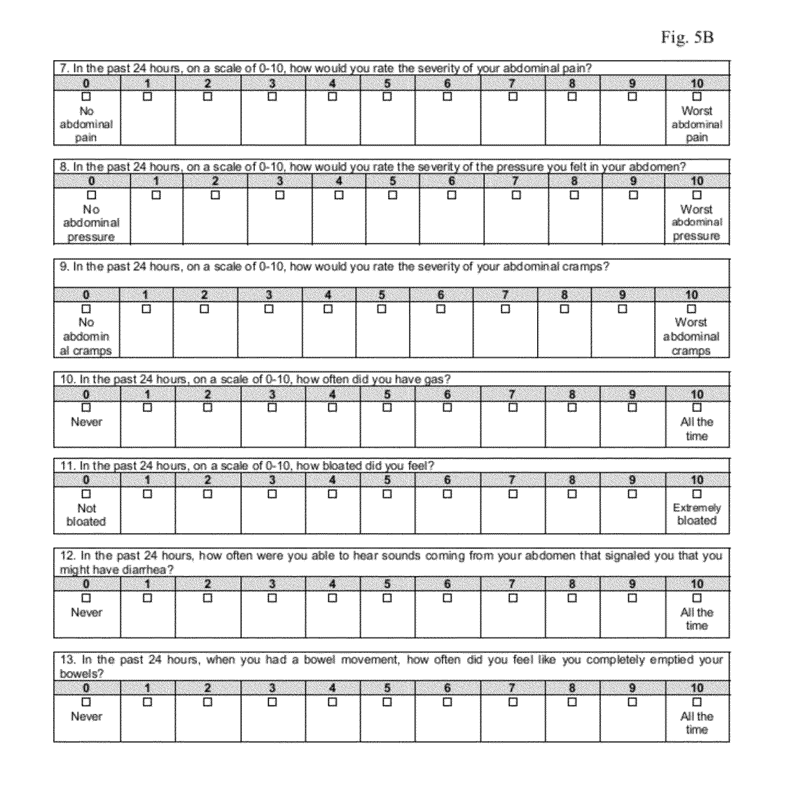

The draft event log (FIG. 4) was also modified after additional analysis, although it continued to require patients to note the date and time of each episode, along with the consistency thereof. For example, a question explicitly asking patients to rate the severity of their diarrhea was deleted on the basis that such conclusion is best drawn from the number and type of diarrhea events. Additionally, a new question was added having a positive/negative response: “Did you feel like you completely emptied your bowels?”

The resulting event log is shown in FIG. 2. The questions provided therein are provided in Table 4.

TABLE 4
Event Log

| Question | Provided responses |
|---|---|
| Which best describes what your | 6 pictures with associated verbal |
| bowel movement looked like? | descriptions according to the Adapted |
| | Bristol Stool Form Scale. |
| Did you feel like you completely | Yes or No |
| emptied your bowels? | |
| How immediate was your need? | A 5-point Likert scale: (1) Not at all; |
| | (2) Slightly; (3) Moderately; (4) |
| | Quite a bit; (5) Extremely. |

The event log and symptom diary (FIGS. 1 and 2) reflect a highly descriptive yet compact tool for assessing a patient's symptoms of IBS-D.

A further refined and preferred event log is as set forth in FIG. 7, which employs a refined 8-level stool scale with associated pictorial and verbal descriptions. The questions provided therein are reflected in Table 5.

TABLE 5
Event Log

| Question | Provided responses |
|---|---|
| Which best describes what your | 8 pictures with associated verbal |
| bowel movement looked like? | descriptions. |
| Did you feel like you completely | Yes or No |
| emptied your bowels? | |
| How immediate was your need? | A 5-point Likert scale: (1) Not at all; |
| | (2) Slightly; (3) Moderately; (4) Quite |
| | a bit; (5) Extremely. |

### Example 2

This example demonstrates the use of the event diary and symptom log to diagnose IBS-D in a patient.

A patient suspected of having IBS-D is provided by a clinician with an event log, such as shown in FIG. 2 or 7. The patient is instructed to record all bowel movements over a set period, e.g., one week or two weeks, as well as to answer the questions provided regarding urgency, emptying of the bowel, and type of bowel movement according to the stool form scale presented in the event log (such as Adapted Bristol Stool Form Scale in FIG. 2 or the eight-point stool form scale shown in FIG. 7).

The clinician also provides the patient with a symptom diary, such as as shown in FIGS. 1A and 1B or 8A and 8B. The patient is instructed to complete a copy of the symptom diary each day for a set period, e.g., one week or two weeks.

Once the patient completes the event log and symptom diaries for the prescribed period, the clinician reviews them. If the patient shows a pattern of response consistent with IBS-D, the patient can be diagnosed with IBS-D.

Accordingly, the event diary and symptom log can be used to diagnose IBS-D in a patient.

### Example 3

This example demonstrates the use of the event diary and symptom log in treating IBS-D in a patient.

A patient undergoing a treatment for IBS-D is provided by a clinician with an event log, such as shown in FIG. 2 or 7. The patient is instructed to record all bowel movements over a set period, e.g., one week or two weeks, as well as to answer the questions provided regarding urgency, emptying of the bowel, and type of bowel movement according to the stool form scale presented in the event log (such as Adapted Bristol Stool Form Scale in FIG. 2 or the eight-point stool form scale shown in FIG. 7).

The clinician also provides the patient with a symptom diary, such as shown in FIGS. 1A and 1B or 8A and 8B. The patient is instructed to complete a copy of the symptom diary each day for a set period, e.g., one week or two weeks.

Once the patient completes the event log and symptom diaries for the prescribed period, the clinician reviews them. If the patient shows improvement based on category score or the total score of six or more items of the symptom diary, then the clinician deems the patient to have improved in IBS symptoms. Alternatively, improvement in the patient's response to two primary endpoints (abdominal pain severity and stool consistency) is considered a favorable response to the IBS-D therapy.

If the patient does not show adequate improvement in IBS symptoms, the clinician revises the dosage and frequency of the IBS treatment, or, optionally, administers a different IBS treatment.

The process of recording IBS events and symptoms in the symptom diary and event log, and evaluating such responses may be repeated until the patient achieves a clinically satisfactory improvement in IBS symptoms.

Accordingly, the event diary and symptom log can be used to evaluate a IBS-D patient's condition and modify treatment accordingly.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of diagnosing irritable bowel syndrome (IBS) comprising:
(a) providing a patient with an event log, wherein the patient is instructed to record in the event log the date and time of each bowel movement together with a description thereof;
(b) reviewing the event log completed by the patient; and
(c) determining whether a diagnosis of irritable bowel syndrome is appropriate.

2. A method of diagnosing irritable bowel syndrome (IBS) comprising:
(a) providing the patient with a symptom diary, wherein the patient is instructed to complete the symptom diary by selecting a characterization from a series of pre-determined characterizations regarding the patient's subjective experience of events associated with IBS;
(b) reviewing the symptom diary completed by the patient; and
(c) determining whether a diagnosis of irritable bowel syndrome is appropriate.

3. A method of diagnosing irritable bowel syndrome (IBS) comprising:
(a) providing a patient with an event log, wherein the patient is instructed to record in the event log the date and time of each bowel movement together with a description thereof;
(b) providing the patient with a symptom diary, wherein the patient is instructed to complete the symptom diary by selecting a characterization from a series of pre-determined characterizations regarding the patient's subjective experience of events associated with IBS;
(c) reviewing the event log and symptom diary completed by the patient; and
(d) determining whether a diagnosis of irritable bowel syndrome is appropriate.

4. A method of treating irritable bowel syndrome (IBS) comprising:
(a) administering to a patient having IBS an IBS treatment at a dosage and frequency;
(b) providing the patient with an event log, wherein the patient is instructed to record in the event log the date and time of each bowel movement together with a description thereof;
(c) reviewing the event log completed by the patient;
(d) revising the dosage and frequency of the IBS treatment, or, optionally, administering a different IBS treatment; and
(e) repeating steps (a)-(d) until the patient achieves a clinically satisfactory improvement in IBS symptoms.

5. A method of treating irritable bowel syndrome (IBS) comprising:
(a) administering to a patient having IBS an IBS treatment at a dosage and frequency;
(b) providing the patient with a symptom diary, wherein the patient is instructed to complete the symptom diary by selecting a characterization from a series of pre-determined characterizations regarding the patient's subjective experience of events associated with IBS;
(c) reviewing the symptom diary completed by the patient;
(d) revising the dosage and frequency of the IBS treatment, or, optionally, administering a different IBS treatment; and
(e) repeating steps (a)-(d) until the patient achieves a clinically satisfactory improvement in IBS symptoms.

6. A method of treating irritable bowel syndrome (IBS) comprising:
(a) administering to a patient having IBS an IBS treatment at a dosage and frequency;
(b) providing the patient with an event log, wherein the patient is instructed to record in the event log the date and time of each bowel movement together with a description thereof; and/or
(c) providing the patient with a symptom diary, wherein the patient is instructed to complete the symptom diary by selecting a characterization from a series of pre-determined characterizations regarding the patient's subjective experience of events associated with IBS;
(d) reviewing the event log and/or symptom diary completed by the patient;
(e) revising the dosage and frequency of the IBS treatment, or, optionally, administering a different IBS treatment; and
(f) repeating steps (a)-(e) until the patient achieves a clinically satisfactory improvement in IBS symptoms.

7. The method of claim 6, wherein the pre-determined characterizations are selected from the group consisting of a 5-point scale (Likert scale), an 11-point scale (0-10), and a positive/negative scale (yes/no).

8. The method of claim 6, wherein the patient is instructed to record bowel movements of one or more weeks in the event log.

9. The method of claim 6, wherein the patient is instructed to record bowel movements of one or more months in the event log.

10. The method of claim 6, wherein the event log further comprises one or more of the following questions:
(a) “How immediate was your need?”;
(b) “Did you feel like you completely emptied your bowels?”;
(c) “Which best describes what your bowel movement looked like?”;
wherein the event log further comprises pre-determined characterizations from which the patient can choose in answering each question.

11. The method of claim 10, wherein 5 different pre-determined characterizations are provided for question (a).

12. The method of claim 10, wherein the pre-determined characterizations for question (b) are positive and negative, or yes and no.

13. The method of claim 10, wherein the pre-determined characterizations for question (c) are based on a scale selected from the Adapted Bristol Stool Form Scale and a scale as depicted in FIG. 7, 9, or 10.

14. The method of claim 10, wherein the event log comprises each of questions (a)-(c).

15. The method of claim 6, wherein the patient is instructed to complete the symptom diary using characterizations of the patient's experience of events associated with IBS on a single day.

16. The method of claim 6, wherein the patient is instructed to complete multiple symptom diaries, wherein each symptom diary is completed using characterizations of the patient's experience of events associated with IBS on a different day.

17. The method of claim 6, wherein the symptom diary comprises one or more of the following questions:
(a) “In the past 24 hours, how would you rate the severity of your stomach pain?”;
(b) “In the past 24 hours, how would you rate the severity of the pressure you felt in your abdomen?”;
(c) “In the past 24 hours, how bloated did you feel?”
(d) “In the past 24 hours, how would you rate the severity of your abdominal cramps?”;
(e) “In the past 24 hours, how would you rate the severity of your rectal spasms?”;
(f) “In the past 24 hours, how would you rate the severity of the burning sensation you experienced after a bowel movement?”;
(g) “In the past 24 hours, how would you rate the severity of your abdominal pain?”
(h) “In the past 24 hours, how often did you have gas?”;
(i)“In the past 24 hours, how often were you able to hear sounds coming from your abdomen that signaled that you might have diarrhea?”; and
(j) “In the past 24 hours, did you have any accidents (lose control of your bowels)?”.

18. The method of claim 17, wherein at least 5 different pre-determined characterizations are provided for each of questions (a)-(i).

19. The method of claim 17, wherein 11 different pre-determined characterizations are provided for each of questions (a)-(g).

20. The method of claim 17, wherein the event log comprises each of questions (a)-(j).

21. The method of claim 20, wherein 11 different pre-determined characterizations are provided for each of questions (a)-(g); 5 different pre-determined characterizations are provided for each of questions (h)-(i); and a positive/negative selection is provided for question (j).

22. The method of claim 6, wherein the treatment comprises a prescription medication approved or under investigational use for treatment of IBS.

23. The method of claim 6, further comprising calculating an index value based on the patient's score in each category of the symptom diary and event log.

24. The method of claim 23, wherein the index value indicates the relative severity of a patient's IBS symptoms as compared to a healthy individual.

25. A method of assessing the symptoms of irritable bowel syndrome (IBS) in a patient comprising providing a human patient suffering from IBS with an event log to record the date and time of events associated with IBS together with a description of the event.

26. The method of claim 25, wherein the event log comprises substantially the form identified in FIG. 2, 4, 6, 7, 9, or 10.

27. A method of assessing the symptoms of IBS in a patient comprising providing a human patient suffering from IBS with symptom diary to select a characterization from a series of pre-determined characterizations regarding the patient's subjective experience of events associated with IBS.

28. The method of claim 27, wherein the symptom diary comprises substantially the form identified in FIG. 1, 3, 5, 8, 11, or 12.

29. The method of claim 25, wherein the patient is undergoing therapy for treatment of IBS.

30. The method of claim 6, wherein the IBS is IBS-D.

31. The method of claim 27, wherein the patient is undergoing therapy for treatment of IBS.

32. The method of claim 6, wherein the symptom diary comprises one or more of the following questions:
(a) “In the past 24 hours, how would you rate the severity of your abdominal pain?”
(b) “In the past 24 hours, how would you rate the severity of your stomach pain?”
(c) “In the past 24 hours, how would you rate the severity of the pressure you felt in your abdomen?”
(d) “In the past 24 hours, how bloated did you feel?”
(e) “In the past 24 hours, how would you rate the severity of your abdominal cramps?”
(f) “In the past 24 hours, how often did you have gas?”
(g) “In the past 24 hours, did you have any accidents (lose control of your bowels)?”

33. The method of claim 6, wherein the event log comprises substantially the form identified in FIG. 2, 4, 6, 7, 9, or 10.

34. The method of claim 27, wherein the symptom diary comprises substantially the form identified in FIG. 1, 3, 5, 8, 11, or 12.

35. The method of claim 32, wherein at least 5 different pre-determined characterizations are provided for each of questions (a)-(f).

36. The method of claim 32, wherein the event log comprises each of questions (a)-(g).

37. The method of claim 20, wherein 10 different pre-determined characterizations are provided for each of questions (a)-(e); 5 different pre-determined characterizations are provided for question (f); and a positive/negative selection is provided for question (g).
